(19) United States
(12) Patent Application Publication
(10) Pub. No.: US 2008/0114457 A1
(43) Pub. Date: May 15, 2008

(54) Methods and devices for connecting implants and devices

(75) Inventors: Boyd Thomas Barker, Bartlett, TN (US); Eric Steven Heinz, Memphis, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic, Inc.

(21) Appl. No.: 11/598,975

(22) Filed: Nov. 14, 2006

(51) Int. Cl. A61F 2/44; A61B 17/08

(52) U.S. Cl. 623/17.16; 606/151

(57) ABSTRACT

A connector device and method are provided to connect at least one implant and at least one device to one another. One or both of the implant and device can be temporary for removal either during the surgery or in a subsequent procedure, or permanently implanted in the body of the patient.

### BACKGROUND

Implants are positioned in a patient for therapeutic, diagnostic, reconstructive, or stabilization purposes, for example. The implants can be positioned at locations deep within the body or at locations where the anatomy is complex, such as along the spinal column. It can be desirable to reference a permanent implant with another permanently implanted device to secure the device in the patient. It may also be desirable to reference a permanent or temporary implant with another temporary device, such as a surgical guidance system referencing device, so that placement of other implants and devices in the patient are referenced to the implant location. There remains a need for systems, devices and methods for securement of implants and devices to one another on temporary and permanent bases.

### SUMMARY

A connector device and method are provided to connect an implant and a device to one another. One or both of the implant and the device can be temporary for removal either during the surgery or in a subsequent procedure, or permanently implanted in the body of the patient. The connector includes an elongated body extending between the implant and the device so that the implant and device are referenced to one another even if the implant is positioned at locations deep within the body of the patient.

According to one aspect, an implant assembly comprises an implant having a height between opposite ends sized to extend between first and second vertebrae of a spinal column and a body sized for positioning in a space between the first and second vertebrae. The implant assembly also includes a device positionable outside the space between the vertebrae. The assembly further includes a connector with an elongated body. The elongated body has a distal end engaged to the implant and a proximal end. The device is engaged along the body adjacent the proximal end. The device is adjustable in position along the body for engagement in any one of a number of positions along the body.

According to another aspect, a spinal implant assembly comprises an implant with a distal portion engageable to a vertebral body and a proximal portion forming a receptacle with an inner thread profile. The assembly further includes a connector including an elongated body extending between a distal end and a proximal end. The elongated body includes a distal portion adjacent the distal end with an external thread profile engaged to the inner thread profile of the first implant. The connector also includes a distal tip extending distally from the external thread profile into the receptacle.

According to another aspect, a spinal implant assembly comprises an implant engageable to the spinal column and a device implantable adjacent the spinal column. The assembly further comprises a connector including an elongated body extending between and connecting the implant to the device. The connector is comprised of substantially electrically non-conductive material and the device is an electromechanical device.

According to one aspect, a method for assembling a spinal construct comprises: positioning an implant between first and second vertebrae; threadingly engaging a distal portion of a connector in a receptacle of the implant; sliding a device along a proximal portion of the connector; and engaging the device to the connector at a selected position along the proximal portion of the connector.

These and other aspects are discussed further below.

### BRIEF DESCRIPTION OF THE DRAWINGS

FIG. 1 is an elevation view of a connector engaged to an implant engaged to bone, with the bone shown diagrammatically in section view.

FIG. 2 is an elevation view of another embodiment connector.

FIG. 3 is an elevation view of another embodiment connector.

FIG. 4 is an elevation view of another embodiment connector.

FIG. 5 is an elevation view of another embodiment connector.

FIG. 6 is an elevation view of another embodiment connector.

FIG. 7 is an elevation view looking in the medial-lateral direction of a spinal column segment with a corpectomy implant and a connector extending therefrom to a posterior stabilization construct.

FIG. 8 is a perspective view of a portion of the corpectomy implant of FIG. 7 with the connector partially shown in hidden lines.

FIG. 9 is a perspective view showing the connector engaged to the corpectomy implant.

FIG. 10 is an elevation view of a portion of the posterior stabilization construct of FIG. 7 looking from the posterior to the anterior direction.

FIG. 11 is a diagrammatic view showing an interbody fusion implant connected to a posterior stabilization construct with the connector of FIG. 1.

FIG. 12 is a diagrammatic view showing an interbody articulating implant connected to a posterior stabilization construct with the connector of FIG. 1.

FIG. 13 is a diagrammatic view of another embodiment device engaged to the connector.

FIG. 14 is a diagrammatic view showing the device and connector of FIG. 13 engaged to an implant secured to a bony portion of the patient.

### DESCRIPTION OF THE ILLUSTRATED EMBODIMENTS

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

A connector is provided that is engageable to at least one implant at a location deep within the body of the patient, and the connector provides an extension for engagement to at least one device that is remotely positioned relative to the at least one implant. Either or both of the implant and the device can be permanently implanted in the patient or removable from the patient after implantation. The connector includes a first end with an engaging portion that is engageable to the at least one implant. The connector includes at least one extension extending from the engaging portion to provide an elongated platform along which one or more devices can be engaged remotely from the implant. The connector links the implant and the device to one another in the patient in one embodiment. In another embodiment, the device is located outside the patient but is connected with the body of the patient via the connector and the implant.

In one embodiment, the implant is a bone anchor such as a bone screw. In a specific embodiment, the bone anchor is a pedicle screw with a threaded shaft and a U-shaped head at a proximal end of the shaft. In another embodiment, the implant is an interbody construct, such as a spinal corpectomy device that replaces one or more vertebrae in the spinal column, a spinal fusion device positioned in a spinal disc space, or an articulating disc type implant that maintains motion of the vertebral levels in which it is implanted. Other embodiments contemplate the implant is a staple, bone bolt, spinal plate, spinal rod, or orthopedic clamp, for example.

In one embodiment, the device is a posterior stabilization construct that includes an elongated stabilization member extending along the spinal column and anchor or fasteners for securing the stabilization member to one or more vertebrae. A coupling mechanism couples the connector to the stabilization element. In another embodiment, the device is any one or combination of a stimulator device, closed container medical device, electromechanical device, or a drug delivery device for chemotherapy or pain management. In another embodiment, the device is a reference frame or guide for a surgical navigation system. The device can be implanted within the patient, or implanted temporarily or permanently outside the body of the patient but connected with the patient through the connector and the implant.

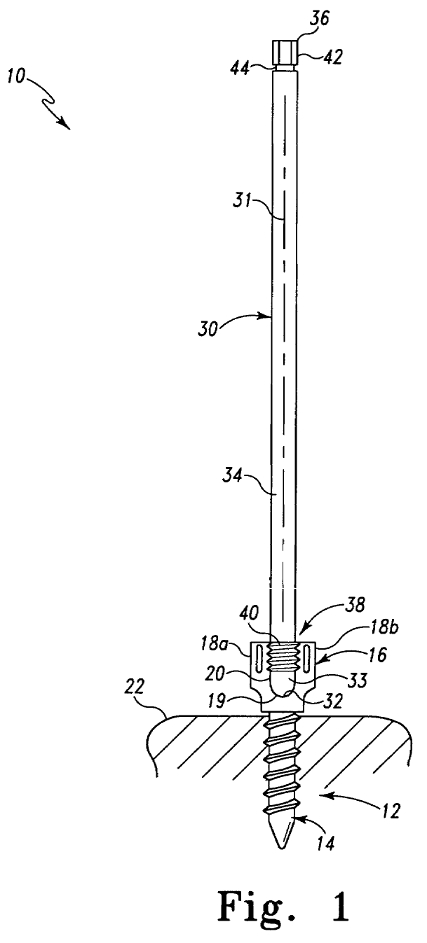

Referring to FIG. 1, there is shown an implant assembly 10 that includes an implant 12 and a connector 30 extending from implant 12. Implant 12 is in the form of a bone screw and includes a distal threaded shaft 14 and a proximal head 16. Threaded shaft 14 is engaged to a bone structure 22. Bony structure 22 can be a vertebral body or other bone of a patient. In one particular procedure, implant 12 is engaged to the pedicle of a vertebra. Head 16 includes opposite axially extending arms 18a, 18b that define a receptacle 20 therebetween. Receptacle 20 opens proximally at the proximal ends of arms 18a, 18b and also opens on opposite sides of arms 18a, 18b to form a transverse passage to receive an elongated implant such as a spinal rod. In other embodiments, receptacle 20 is closed on the sides thereof and forms a single opening therein into which connector 30 is positioned. The bottom or distal surface of receptacle 20 is formed by a concavely curved surface 19 extending between arms 18a, 18b.

Connector 30 includes a distal end 32 and an elongated body 34 extending proximally from distal end 32 to a proximal end 36 along longitudinal axis 31. Body 34 includes a distal portion 38 having an outer thread profile 40 extending along a portion of the length of body 34. Distal end 32 includes a rounded, bullet shaped tip 33 to positively seat against convex bottom surface 19 of head 16. Tip 33 includes a non-threaded, smooth outer surface profile extending distally from thread profile 40 to provide an intimate fit with the non-threaded portion of arms 18a, 18b. Thread profile 40 is engageable to internal threads along arms 18a, 18b, while the rounded distal end provides an intimate fit in receptacle 20. In one embodiment, the entire periphery of the portion of connector 30 engaged in receptacle 20 of first implant 12 is in contact with first implant 12, providing connector 30 with a stable engagement relationship with first implant 12 that minimizes rocking, pivoting and relative movement with implant 12.

Proximal end 36 of connector 30 includes a tool engaging portion 42. In the illustrated embodiment, tool engaging portion 42 is in the form of a hexagonal head that positively engages a tool (not shown) that is employed to rotate connector 30 and seat it in implant 12. Connector 30 also includes a break-off portion 44 between body 34 and tool engaging portion 42. Break-off portion 44 provides a reduced thickness that allows tool engaging portion 44 to be severed from body 34 upon application of sufficient torque. In another embodiment, a break-off portion is provided adjacent thread profile 40 so that body 34 can be removed adjacent the implant 12 while thread profile 40 is engaged to implant 12. In this embodiment, connector 30 also functions as a retaining device to provide a function post-operatively while engaged to implant 12. The distal break-off portion can be provided in addition to or alternatively to the proximal break-off portion 44. In still other embodiment, body 34 is severed by a cutting tool adjacent thread profile 40.

Other embodiments contemplate other configurations for tool engaging portion 42. In one embodiment tool engaging portion 42 includes an internal hex or other non-circular configuration to positively engage a tool. In another embodiment, tool engaging portion 42 includes an external non-circular shape to positively engage a tool. In another embodiment, tool engaging portion 42 is circular or of other external shape that is the same as the external shape of body 34.

When engaging connector 30 to implant 12, a counter-torque instrument can be engaged to implant 12 while engaging connector 30 to implant 12 to prevent implant 12 from rotating while connector 30 is secured thereto. The counter-torque instrument, in one embodiment, includes a tubular body that extends around body 34 of connector 30 and includes a distal end that fits around arms 18a, 18b in a manner that prevents rotation of the implant relative to the counter-torque instrument. A handle or proximal portion of the tubular body is held or secured to maintain the positioning of the counter-torque instrument and implant 12 as connector 30 is engaged to implant 12. In another embodiment, the counter-torque instrument is provided in a pliers type arrangement with a distal end that grippingly engages one or both of the arms 18a, 18b and a proximal handle that is held to prevent rotation of implant 12 during engagement of connector 30.

In FIG. 1 body 34 is in the form of an elongated rod-like member with a circular cross-section orthogonally to longitudinal axis 31. Body 34 is linear along longitudinal axis 31 and includes a constant cross-sectional dimension between proximal and distal ends 36, 32 to form a circular cylinder, i.e. a cylinder with a circular cross-section. Body 34 includes a smooth outer surface extending therearound to facilitate movement of connection devices or implants along body 34 for engagement at any selected position therealong. Other embodiments contemplate that outer surface 34 includes knurlings, ridges, teeth or stepped regions to provide structure for positive engagement by a connection device or other implant.

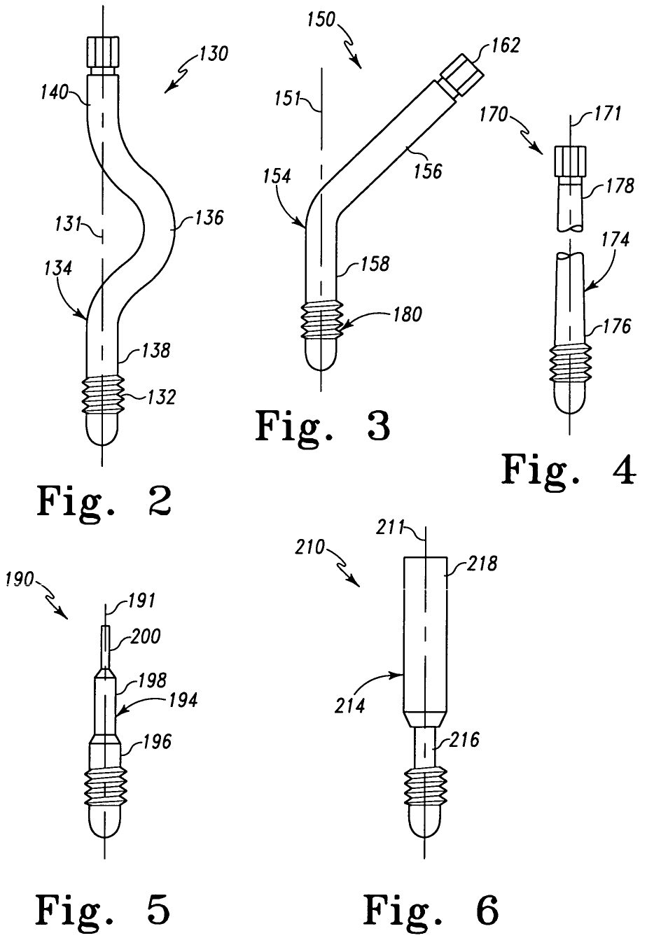

Various embodiments are contemplated where body 34 includes other forms. For example, FIG. 2 there is shown connector 130 that is identical to connector 30 except for the shape of body 134. Body 134 includes an offset portion 136 having a U-shape, and opposite end portions 138, 140 extending axially from offset portion 136 along longitudinal axis 131. End portion 138 includes external thread profile 132 for engagement with a receptacle of an implant. Offset portion 136 has a curved shape to form a concave curvature with longitudinal axis 131 while providing smooth transitions between the offset portion 136 and end portions 138, 140. Other embodiments contemplate angular transitions that provide abrupt changes in profile along longitudinal axis 131. In FIG. 3 there is shown connector 150 that is identical to connector 30 except for the shape of body 154. Body 154 includes an offset proximal end portion 156 obliquely oriented to longitudinal axis 151, which extends along distal portion 158. Offset portion 156 orients proximal end 162 at a location offset from longitudinal axis 151. Offset portion 156 is joined to distal portion 158 with an angular bend. Distal portion 158 includes external thread profile 160 for engagement in the receptacle of a first implant. Other embodiments contemplate multiple bends to provide multiple offset portions along body 154.

In FIGS. 4-6 other embodiments of connector 30 are provided where the cross-section varies along the length of the connector body. For example, connector 170 in FIG. 4 includes a body 174 extending along a longitudinal axis 171. Body 174 is tapered from distal end portion 176 toward proximal end portion 178. In FIG. 5 connector 190 includes body 194 extending along longitudinal axis 191. Body 194 has a distal portion 196, an intermediate portion 198, and a proximal portion 200. The cross-sectional size of the portions 196, 198, 200 reduce proximally in a step-wise manner so that proximal portion 200 is smaller than intermediate portion 198 and distal portion 196 is greater in size than intermediate portion 198. Portions 196, 198, 200 form circular cylinder shapes along their respective lengths. Other embodiments contemplate more than three portions of differing sizes. In yet another embodiment, proximal portion 200 is provided with a size greater than intermediate portion 198. The reduced size intermediate portion provides a location for bending or flexing of connector 190 to orient proximal portion 200 in a desired position. In FIG. 6 connector 210 includes a body 214 extending along longitudinal axis 211. Body 214 includes a distal portion 216 that has a cross-sectional size that is less than the cross-sectional size of proximal portion 218.

In each of the connector embodiments discussed herein, the bodies include a circular cross-section. It is also contemplated that the bodies can include cross-sections that are non-circular. Furthermore, the cross-sectional shapes can vary along the length of the connector body. The connector bodies can be made from metal or metal alloys, such as aluminum, stainless steel, titanium, or shape memory materials. It is also contemplated that the bodies can be made from polymer material, such as polyetheretherketone (PEEK), plastics, ceramics, elastomers, or other suitable biocompatible material. The bodies are rigid in one embodiment so that the configuration is constant. Other embodiments contemplate the body is bendable to a desire shape or orientation. The bodies can also be formable to a desired configuration.

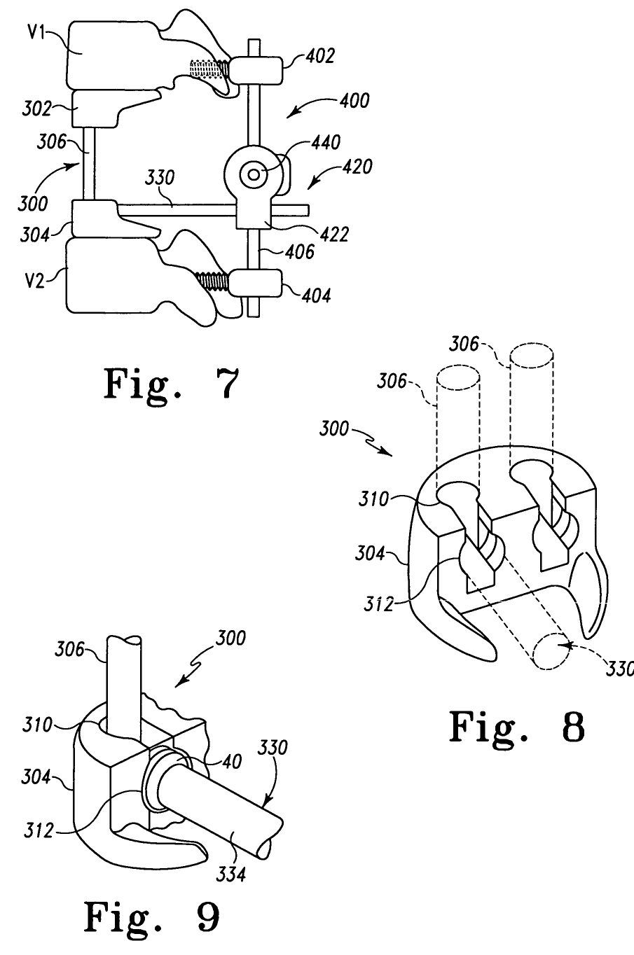

Referring now to FIG. 7, there is shown an implant assembly with connector 330, which can be any embodiment connector 30, 130, 150, 170, 190, 210 and variations thereof discussed herein. The implant assembly includes an implant 300 in the form of a corpectomy device positioned between first and second vertebrae V1 and V2. One or more vertebrae and associated spinal discs have been removed to accommodate placement of first implant 300. Connector 330 is engaged adjacent its distal end with implant 300, and a device 400 is engaged adjacent the proximal end of connector 330. Device 400 is a posterior spinal stabilization construct engaged to vertebrae V1 and V2. Connector 330 links implant 300 with device 400 and provides post-operative stability and maintenance of the positioning of the implant 300.

Implant 300 includes upper base 302 and a lower base 304 positioned adjacent receptive ones of the vertebrae V1 and V2. Implant 300 includes one or more axial struts 306 connecting bases 302, 304 to provide transfer of the loads from vertebra V1 to vertebra V2. Device 400 includes a first bone engaging fastener 402 engaged to first vertebra V1 and a second bone engaging fastener 404 engaged to second vertebra V2. An elongate member 406 extends between and is engaged to fasteners 402, 404 to provide stabilization outside the disc space along the posterior of the spinal column. In other embodiments, fasteners and an elongate member are engaged along the vertebrae V1, V2 on the contra-lateral side of the spinal column. It is also contemplated that the elongate elements can extend along and be engaged to more than two vertebrae. Elongate member 406 is shown in the Figures in the form of a spinal rod. Other embodiments contemplate other forms for elongate member 406, including the form of a plate, tether, cable, or staple, for example.

As shown in FIGS. 8 and 9, connection of the connector 330 with base 304 is shown. Base 304 includes an axial receptacle 310 extending therein to receive strut 306. A transverse receptacle 312 extends transversely to receptacle 310 and opens posteriorly in base 304. Connector 330 is positioned with its distal end in transverse receptacle 312, and with its external thread profile engaged to internal threads along transverse receptacle 312. The distal end can contact strut 306 and firmly seat it in base 304 to maintain it in position therein. It is also contemplated that a second connector can be engaged to base 304 to secure the other strut 306 in base 304. Alternatively, a set screw or other device can be engaged in base 304 to secure the one or more other struts in base 304.

Connector 330 is engaged to elongate member 406 with a coupling mechanism 420. One example of a coupling mechanism is shown in FIGS. 7 and 10, it being understood that any suitable coupling mechanism for securing connector 330 to elongate member 406 is contemplated. Elongate member 406 is secured to the respective bone engaging fasteners 402, 404 with an engaging member, such as set screw 408. Clamping member 422 includes a passage 424 through which connector 330 extends and a pair of arms 426, 428 extending from passage 424. The location of clamping member 422 along the proximal portion of connector 330 can be adjusted to accommodate the spacing between implants 300, 400. Coupling mechanism 420 further includes a mounting member 430 through which elongate member 406 extends.

Arm 428 includes a contact surface 432 positioned adjacent a contact surface 434 of mounting member 430. Contact surfaces 432, 434 are provided with interdigitating radial splines that secure mounting member 430 and clamping member 422 in a selected orientation relative to one another. Fastener 440 extends through arms 426, 428 and engages mounting member 430 with an end 442. In one embodiment, end 442 is threadingly engaged to mounting member 430 and includes an opposite shoulder or head 444 that contacts arm 426 to compress arms 426, 428 toward one another and seat arm 428 against mounting member 430 at contact surfaces 432, 434. In the clamped arrangement, arms 426, 428 are clamped around connector 330 and clamping member 422 is secured to mounting member 430. Mounting member 430 can axially float along elongate member 406, or can be secured in position in mounting member 430 by a set screw or by the end of fastener 440 contacting elongate member 406. Any length of connector 330 extending proximally from clamping member 422 can be removed by cutting or severing it with a cutting tool (not shown) to minimize intrusion into adjacent tissue.

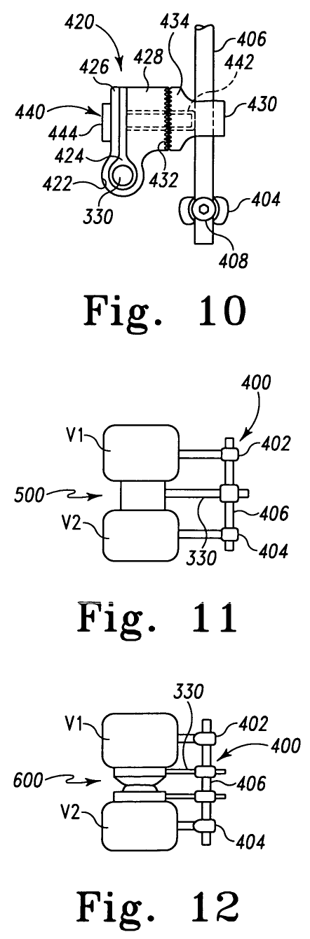

Referring now to FIG. 11, there is shown another embodiment implant assembly with an implant in the space between vertebrae V1 and V2 in the form of a fusion device 500. Fusion device 500 is sized for positioning in a spinal disc space between vertebrae V1 and V2. Fusion device includes a height between upper and lower vertebral contacting surfaces sized to contact the vertebrae V1, V2 and support the vertebrae at a desired spacing during fusion between the vertebrae. Connector 330 is engaged to device 500 adjacent its distal end and extends proximally from the device 500 to device 400.

Referring now to FIG. 12, there is shown another embodiment implant assembly with an implant in the space between vertebrae V1 and V2 in the form of an articulating device 600. Articulating device 600 is sized for positioning in the spinal disc space between vertebrae V1 and V2, and includes an upper portion 602 adjacent vertebra V1 that is movable relative to a lower portion 604 adjacent vertebra V2. The articulating portions 602, 604 preserve motion of the vertebral level or levels in which device 600 is positioned. First and second connectors 330 are engaged to respective ones of the upper and lower portions 602, 604. Connectors 330 extend proximally from the device 600 to device 400. In other embodiments, the implant assembly includes a single connector 330 engaged to one of the portions 602, 604.

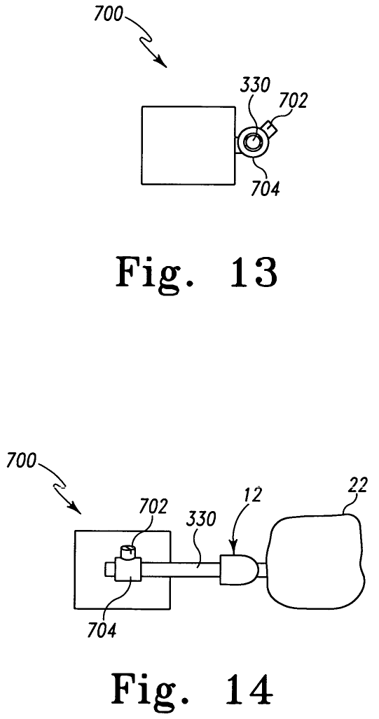

FIGS. 13 and 14 show another embodiment implant assembly with a device 700 engaged to implant 12 with connector 330. Implant 12 is engaged to bony structure 22. Device 700 is in the form of an implantable electromechanical treatment device, such as a monitor, drug delivery device, electro-therapy device, stimulation device, treatment device, or other implantable treatment device. The positioning of device 700 in the patient is maintained since it is anchored to the bone with implant 12 and connector 330. Device 700 includes a mounting member 704 to receive connector 330, and the location of implant 700 along connector 330 can be varied and then maintained with an engaging member, such as set screw 702, extending through mounting member 704 to contact connector 330. It should be understood that implant 12 could be any of the other implant embodiments discussed herein.

A method of using one or more connectors discussed herein will now be described. The surgical methods for implanting the connectors can be employed in open surgical procedures where skin and tissue is retracted, and in minimally invasive surgical procedures where the connector and the one or more implants engaged by the connector are positioned in the patient with one or more minimally invasive access approaches formed by micro-incisions, retractors, sleeves, or expanding sleeves, for example.

In one procedure, the implant is positioned in the patient under fluoroscopy and then the connector is engaged to the implant. The device is engaged to the proximal portion of the connector and referenced to the structure in the patient to which the implant is engaged. In other procedures, the connector is manipulated to provide derotation of the spinal column, to move bony structures into alignment, or other reduction or derotation procedure. After derotation, the connector is removed and spinal rods, plates or other devices are engaged to one or more of the implants to maintain the spinal column in the desired orientation. Accordingly, the connector allows the surgeon to employ the same implants for derotation and stabilization.

In one procedure, a surgeon will make an incision into the patient at a place relatively proximate to one or more vertebral levels or other bone(s) to which the implant is to be attached. After the appropriate access to the surgical site is obtained, the bone is instrumented (e.g. the pedicle of a vertebra or removal of one or discs and/or vertebrae)) is prepared in a standard manner. For example, an awl or drill may be used to prepare a hole, which is then probed for depth and tapped if appropriate for an implant in the form of a pedicle screw. The one or more implants are then inserted into the hole in the bone or into the space between adjacent bones. A connector is engaged to a receptacle of the at least one implant. In one embodiment, the connectors are pre-bent or bent during surgery to include a curvature, for example, to replicate or extend around anatomy of the spinal column. Additional connectors are engaged to the implant or other implants as may be necessary. A device is then engaged to the opposite end of the connector and physically and mechanically referenced to the implant via the at least one connector. The same device can be engaged to multiple connectors. It is also contemplated that multiple devices can be referenced to the same implant either along the same connector or with multiple connectors engaged to multiple devices. It is also contemplated that multiple implants are connected to multiple devices with one or more connectors. In still another procedure, multiple implants are connected to a single device with multiple connectors.

After connecting the implant and the device, the device is readied for further procedures. For example, in the situation where the device s a stabilization construct, the incision is closed and the device is post-operatively linked with the implant to together provide a desired stabilization effect. In situations where the device is an electromechanical device, the device provides post-operative therapy, stimulation or other effect while its positioning in the patient is maintained by its connection with the implant via the connector. The device, connector and the implant can remain in the patient post-operatively and can be removed in a subsequent procedure. Other embodiments contemplate that the implant, the device and connector are permanently implanted in the patient. In still other procedures, the connector and one or both of the implant and the device are implanted for temporary purposes and removed in the same surgical procedure.

It will further be appreciated that the embodiments for the connector described above should be made of materials suitable for implantation within the human or other body, and may consist of or be comprised of inert metals like titanium or stainless steel. Other sturdy materials such as certain ceramics or plastics may also be considered. Bio-resorbable materials, such as polylactic acid compounds, may be used along with or as a part of the parts described above. In one embodiment, a connector made of polyethetherketone (PEEK) is employed. In still other embodiments, the connector is made from non-conductive material so that when engaged to an electromechanical or electrical implant electrical current from the device is not conducted by the connector. In other procedures the device is temporary and employed as a reference guide for installation of one or more other implants relative to the implant. For example, the device is a reference frame for a surgical navigation system in one embodiment, or a mechanical guide that guides the positioning of additional implants into the patient relative to the first implant in another embodiment.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An implant assembly, comprising:
an implant having a height between opposite ends sized to extend between first and second vertebrae of a spinal column, said implant further including a body sized for positioning in a space between the first and second vertebrae;
a device positionable outside the space between the vertebrae; and
a connector including an elongated body with a distal end engaged to said implant and a proximal end, said device engaged along said body adjacent said proximal end, wherein said device is adjustable in position along said body for engagement in any one of a number of positions along said body.

2. The assembly of claim 1, wherein said connector includes a distal portion with an outer thread profile and said implant includes a receptacle, said thread profile threadingly engaging said receptacle.

3. The assembly of claim 2, wherein said distal portion of said connector includes a convexly rounded tip extending distally from said thread profile to said distal end.

4. The assembly of claim 1, wherein said elongated body forms a circular cylinder extending along a longitudinal axis between said distal end and said proximal end.

5. The assembly of claim 4, wherein said body includes a smooth outer surface extending from said proximal end to a thread profile adjacent said distal end.

6. The assembly of claim 1, wherein said elongated body includes a distal portion extending along a longitudinal axis and a proximal portion that is offset from said longitudinal axis.

7. The assembly of claim 6, wherein said proximal portion is connected to said distal portion with a bend and said proximal portion is obliquely oriented to said longitudinal axis.

8. The assembly of claim 6, wherein said proximal portion includes a curved portion forming a concavely curved offset to said longitudinal axis.

9. The assembly of claim 8, wherein said proximal portion includes a proximal end portion aligned along said longitudinal axis.

10. The assembly of claim 1, wherein said elongated body extends along a longitudinal axis and defines a tapered cross-section along said longitudinal axis.

11. The assembly of claim 1, wherein said elongated body extends along a longitudinal axis, said body including a distal portion having a first cross-sectional dimension and a second portion having a second cross-sectional dimension, said first and second cross-sectional dimensions differing from one another.

12. The assembly of claim 11, wherein said distal portion forms a circular cylinder and said second portion forms a circular cylinder.

13. The assembly of claim 12, wherein said body further includes an intermediate portion forming a circular cylinder along said longitudinal axis between said first portion and said second portion, said third portion having a cross-sectional dimension that differs from said first and second cross-sectional dimensions.

14. The assembly of claim 13, wherein said first cross-section dimension is greater than said third cross-section dimension and said third cross-sectional dimension is greater than said second cross-sectional dimension.

15. The assembly of claim 1, wherein said implant includes a first base portion adjacent the first vertebra and a second base portion adjacent the second vertebra, and said connector is engaged to one of said first and second base portions in a receptacle thereof.

16. The assembly of claim 15, wherein said implant includes at least one strut extending between said first and second base portions, said at least one strut including an end in said receptacle and said connector restrains said strut in said receptacle when engaged in said receptacle.

17. The assembly of claim 16, wherein said device includes first and second fasteners engageable to respective ones of the first and second vertebrae and an elongate member extending between and engaged to said first and second fasteners, said connector being engaged to said elongate member with a coupling mechanism.

18. The assembly of claim 17, wherein said coupling mechanism is adjustable in position along said body of said connector and along said elongate member.

19. The assembly of claim 15, wherein said first and second base portions are movably coupled to one another.

20. The assembly of claim 1, wherein the space is formed by a single spinal disc space between the first and second vertebrae.

21. The assembly of claim 1, wherein the space is formed by at least one vertebral body removed from between the first and second vertebrae.

22. A spinal implant assembly, comprising:
an implant with a distal portion engageable to a vertebral body and a proximal portion forming a receptacle with an inner thread profile; and
a connector including an elongated body extending between a distal end and a proximal end, said elongated body including a distal portion adjacent said distal end with an external thread profile engaged to said inner thread profile of said implant, said connector further including a distal tip extending distally from said external thread profile into said receptacle.

23. The assembly of claim 22, wherein said receptacle of said implant includes a concavely curved bottom surface and said distal tip includes a convexly curved distal end positioned in contact with said bottom surface when said external thread profile of said connector is engaged to said inner thread profile of said implant.

24. The assembly of claim 23, wherein said distal portion of said implant includes an outer thread profile and said receptacle is formed by a pair of arms extending along said receptacle from said bottom surface, said bottom surface extending between said pair of arms.

25. The assembly of claim 24, wherein said elongated body forms a circular cylinder extending along a longitudinal axis between said external thread profile and said proximal end.

26. The assembly of claim 22, wherein said body includes a smooth outer surface extending proximally from said external thread profile to said proximal end.

27. The assembly of claim 22, wherein said distal portion of said elongated body extends along a longitudinal axis and said elongated body includes a portion proximally of said distal portion that is offset from and obliquely oriented to said longitudinal axis.

28. The assembly of claim 22, wherein said elongated body includes a proximal portion adjacent said proximal end that is connected to said distal portion with a bend with said bend offset from said longitudinal axis and with said proximal and distal portions aligned along said longitudinal axis.

29. The assembly of claim 28, wherein said bend forms a concavely curved offset to said longitudinal axis.

30. The assembly of claim 22, further comprising a device engaged to said elongated body adjacent said proximal end.

31. The assembly of claim 30, wherein said device is a surgical navigation reference device.

32. A spinal implant assembly, comprising:
an implant engageable to the spinal column;
a device implantable adjacent the spinal column; and
a connector including an elongated body extending between and connecting said implant to said device, wherein said connector is comprised of substantially electrically non-conductive material and said device is an electromechanical device.

33. The assembly of claim 32, wherein:
said implant is a bone screw with a distal threaded portion engageable to a vertebral body and a proximal portion with a receptacle; and
said connector includes a distal end portion threadingly engaged to said implant in said receptacle.

34. The assembly of claim 33, wherein said distal portion of said connector includes a convexly curved distal end bottomed in said receptacle of said implant.

35. The assembly of claim 34, wherein said convexly curved distal end is formed along a bullet-shaped tip of said elongated body that extends distally from an externally threaded portion of said elongated body that is engaged to said receptacle of said implant.

36. The assembly of claim 32, wherein said connector includes an elongated body extending proximally from said implant and said device is engageable at any one of a number of positions along said elongated body.

37. A method for assembling a spinal construct, comprising:
positioning an implant between first and second vertebrae;
threadingly engaging a distal portion of a connector in a receptacle of the implant;
sliding a device along a proximal portion of the connector; and
fixing the device to the connector at a selected position along the proximal portion of the connector at a location remote from the first and second vertebrae.

38. The method of claim 37, wherein threadingly engaging the distal portion includes positioning a distal end of the connector in contact with a strut of the implant, the strut extending between the first and second vertebrae.

39. The method of claim 38, wherein the implant is a corpectomy device and includes a first base portion adjacent the first vertebra and a second base portion adjacent the second vertebra, and the strut extends between the first and second base portions.

40. The method of claim 37, wherein positioning the implant includes positioning the implant in a spinal disc space between adjacent first and second vertebrae.

41. The method of claim 40, wherein the implant is a spinal fusion device.

42. The method of claim 40, wherein the implant is an articulating disc implant.

43. The method of claim 37, further comprising removing a portion of the connector extending proximally from the device.
